(19) United States
(12) Patent Application Publication
(10) Pub. No.: US 2003/0220259 A1
(43) Pub. Date: Nov. 27, 2003

(54) Treatment of neurological disorders

(76) Inventors: Robbert Benner, Barendrecht (NL); Nisar Ahmed Khan, Rotterdam (NL); Gert Wensvoort, Koekange (NL)

Correspondence Address: TRASK BRITT, P.O. BOX 2550, SALT LAKE CITY, UT, 84110, US

(21) Appl. No.: 10/409,654

(22) Filed: Apr. 8, 2003

Related U.S. Application Data
(63) Continuation-in-part of application No. 10/028,075, filed on Dec. 21, 2001.

(51) Int. Cl. A61K 48/00; A61K 38/17

(52) U.S. Cl. 514/12; 514/44

(57) ABSTRACT

The invention relates to the treatment of the inflammatory component of neurological disorders or so called neuroimmune disorders such as schizophrenia, manic depression and other bipolar disorders, multiple sclerosis, postpartum psychosis and autism. The invention provides a method for modulating a neurological disorder in a subject comprising providing the subject with a gene-regulatory peptide or functional analogue thereof. The invention also provides use of an NF-κB down-regulating peptide or functional analogue thereof for the production of a pharmaceutical composition for the treatment of a neurological disorder.

### CROSS-REFERENCE TO RELATED APPLICATION

This application is a continuation-in-part of U.S. patent application Ser. No. 10/028,075, filed Dec. 21, 2001, pending, the content of the entirety of which is incorporated by this reference.

### TECHNICAL FIELD

The current invention relates to the body's innate way of modulation of important physiological processes and builds on insights reported in PCT International Publications WO99/59617 and WO01/72831 and PCT International Patent Application PCT/NL02/00639.

### BACKGROUND

In the aforementioned patent applications, small gene-regulatory peptides are described that are present naturally in pregnant women and are derived from proteolytic breakdown of placental gonadotropins such as human chorionic gonadotropin (hCG) produced during pregnancy. These peptides (in their active state often only at about 4 to 6 amino acids long) were shown to have unsurpassed immunological activity that they exert by regulating expression of genes encoding inflammatory mediators such as cytokines. Surprisingly, it was found that breakdown of hCG provides a cascade of peptides that help maintain a pregnant woman's immunological homeostasis. These peptides are nature's own substances that balance the immune system to assure that the mother stays immunologically sound while her fetus does not get prematurely rejected during pregnancy but instead is safely carried through its time of birth.

Where it was generally thought that the smallest breakdown products of proteins have no specific biological function on their own (except to serve as antigen for the immune system), it now emerges that the body in fact routinely utilizes the normal process of proteolytic breakdown of the proteins it produces to generate important gene-regulatory compounds, short peptides that control the expression of the body's own genes. Apparently, the body uses a gene-control system ruled by small broken down products of the exact proteins that are encoded by its own genes.

It is long known that during pregnancy the maternal system introduces a status of temporary immuno-modulation which results in suppression of maternal rejection responses directed against the fetus. Paradoxically, during pregnancy, often the mother's resistance to infection is increased and she is found to be better protected against the clinical symptoms of various auto-immune diseases such as rheumatism and multiple sclerosis. The protection of the fetus can thus not be interpreted only as a result of immune suppression. Each of the above three applications have provided insights by which the immunological balance between protection of the mother and protection of the fetus can be understood.

It was shown that certain short breakdown products of hCG (i.e., short peptides which can easily be synthesized, if needed modified, and used as pharmaceutical composition) exert a major regulatory activity on pro- or anti-inflammatory cytokine cascades that are governed by a family of crucial transcription factors, the NFκB family which stands central in regulating the expression of genes that shape the body's immune response.

Most of the hCG produced during pregnancy is produced by cells of the placenta, the exact organ where cells and tissues of mother and child most intensely meet and where immuno-modulation is most needed to fight off rejection. Being produced locally, the gene-regulatory peptides which are broken down from hCG in the placenta immediately balance the pro- or anti-inflammatory cytokine cascades found in the no-mans land between mother and child. Being produced by the typical placental cell, the trophoblast, the peptides traverse extracellular space; enter cells of the immune system and exert their immuno-modulatory activity by modulating NFκB-mediated expression of cytokine genes, thereby keeping the immunological responses in the placenta at bay.

### BRIEF SUMMARY OF THE INVENTION

It is herein postulated that the beneficial effects seen on the occurrence and severity of auto-immune disease in the pregnant woman result from an overspill of the hCG-derived peptides into the body as a whole; however, these effects must not be overestimated, as it is easily understood that the further away from the placenta, the less immuno-modulatory activity aimed at preventing rejection of the fetus will be seen, if only because of a dilution of the placenta-produced peptides throughout the body as a whole. However, the immuno-modulatory and gene-regulatory activity of the peptides should by no means only be thought to occur during pregnancy and in the placenta; man and women alike produce hCG, for example in their pituitaries, and nature certainly utilizes the gene-regulatory activities of peptides in a larger whole.

Consequently, a novel therapeutic inroad is provided, using the pharmaceutical potential of gene-regulatory peptides and derivatives thereof. Indeed, evidence of specific up- or down-regulation of NFκB driven pro- or anti-inflammatory cytokine cascades that are each, and in concert, directing the body's immune response was found in silico in gene-arrays by expression profiling studies, in vitro after treatment of immune cells and in vivo in experimental animals treated with gene-regulatory peptides. Also, considering that NFκB is a primary effector of disease (A. S. Baldwin, J. Clin. Invest., 2001, 107:3-6), using the hCG derived gene-regulatory peptides offer significant potential for the treatment of a variety of human and animal diseases, thereby tapping the pharmaceutical potential of the exact substances that help balance the mother's immune system such that her pregnancy is safely maintained.

### DETAILED DESCRIPTION OF THE INVENTION

The invention in particular relates to the treatment of neurological disorders or so called neuroimmune disorders such as schizophrenia, manic depression and other bipolar disorders, multiple sclerosis, post-partum psychosis, autism, chronic fatigue syndrome (CFS), fibromyalgia, Alzheimer's, mood disorders and certain forms of stress. Although there are major differences in etiology and mechanisms of pathogenesis of each of these syndromes and or diseases, there are in fact common inflammatory and immunomodulatory pathways that are shared within the pathogenesis of neurological disorders.

Evidence of immune abnormalities in patients suffering from psychological disease clearly shows the implication of the immune system in pathogenesis. Neuroimmune disorders have become recognized as common pathogenetic factors in the development of psycho- or neuropathologies. The neurochemical and immunologic findings indicate multiple pathways of the pathogenesis; herein, we discuss the role of inflammatory disease in neurological disorders. For example, chronic fatigue syndrome is a condition that affects women in disproportionate numbers, and that is often exacerbated in the premenstrual period and following physical exertion. The signs and symptoms, which include fatigue, myalgia, and low-grade fever, are similar to those experienced by patients infused with cytokines such as interleukin-1. In general, during the development of a neuroimmune disorder, the TNF-α family and other pro-inflammatory cytokines are highly elevated in cerebrospinal fluid (CSF), demonstrative of foci of inflammation in the brain leading to an array of destructive and degenerative responses directed at diverse areas in the CNS. Major mood disorders are leading causes of disability from early adolescence onward and leading sources of disease burden, surpassing cardiovascular diseases, dementia, lung cancer and diabetes. As the, there is a major role for inflammatory cytokines and immune cells in the pathophysiology of mood disorders, it was recently also found that T cells and monocytes function at a higher, pro-inflammatory level in patients with bipolar disorder. Successful therapy of these destructive and degenerative disorders that affect the adult human central nervous system (CNS) will require the ability both to reduce the rate and extent of tissue injury, and to restore or replace destroyed tissue. Neuroimaging studies have shown that functional organization occurs spontaneously in the adult human brain in response to tissue insults. The extent of this compensatory mechanism may be limited, necessitating development of active methods of intervention. Replacement of a single neurotransmitter, neurohormone or trophic factor may suffice if the injury is limited or affected as suppression or altered pathway within the CNS through proinflammatory regulators. The hippocampus is a source for mitotically active neuronal progenitor cells which can hypothetically replace neurons and myelinating cells. It is the control of these cells and the health and activity of other cells which offers new insight and hope of treating heretofore chronic CNS disease. It is areas such as cells in the adult human dentate gyrus which may be part of the key to controlling immunomodulation and growth support of the brain and its diverse functions which span from memory and cognition to its endocrine and immunologic activities .As with all complex traits, a neurological disorder results from an interplay between as yet unidentified environmental factors and susceptibility genes. Together, these factors trigger a cascade of events, involving engagement of the immune system, acute inflammatory injury of the central nervous system, notably axons and glia, recovery of function and structural repair, post-inflammatory gliosis, and neurodegeneration. The sequential involvement of these processes underlies the clinical course characterised by episodes with recovery interchanged with episodes leaving persistent deficits, episodes which we generally call psychological disorders.

For a more detailed example, although there are several forms of autism (which often present themselves already at birth) which may have clear genetic etiologies, the most common forms however occur long after normal births and are associated with proinflammatory cytokine dysregulation. According to recent epidemiological surveys, autistic spectrum disorders have become recognized as common childhood psychopathologies. These life-lasting conditions demonstrate a strong genetic determinant consistent with a polygenic mode of inheritance for which several autism susceptibility regions have been identified. Parallel evidence of immune abnormalities in autistic patients argues for an implication of the immune system in pathogenesis. This introduction summarizes advances in the molecular genetics of autism, as well as recently emerging concerns addressing the disease incidence and triggering factors. The neurochemical and immunologic findings are analyzed in the context of a neuroimmune hypothesis for specific neurologic disorders. For example, pregnancy and the post partum period are important modulators of the immune system and the immune suppression in pregnancy is followed by an immune activation in the puerperium. In another example, autism is influenced by specific food allergies or even the early use of vaccines which may cause changes in the regulation of innate or acquired immunity and set up neuroendocrine dysfunction. Also, neurological disorders are often associated with autoimmune disorders in the patients' relatives. Comi A. M. et al. (J. Child Neurol. June 1999; 14(6):388-94) evaluated the frequency of autoimmune disorders, as well as various prenatal and postnatal events in autism, and surveyed the families of 61 autistic patients and 46 healthy controls using questionnaires. The mean number of autoimmune disorders was greater in families with autism; 46% had two or more members with autoimmune disorders. As the number of family members with autoimmune disorders increased from one to three, the risk of autism was greater, with an odds ratio that increased from 1.9 to 5.5, respectively. In mothers and first-degree relatives of autistic children, there were more autoimmune disorders (16% and 21%) as compared to controls (2% and 4%), with odds ratios of 8.8 and 6.0, respectively. The most common autoimmune disorders in both groups were type 1 diabetes, adult rheumatoid arthritis, hypothyroidism, and systemic lupus erythematosus. Forty-six percent of the autism group reported having relatives with rheumatoid diseases, as compared to 26% of the controls. Prenatal maternal urinary tract, upper respiratory and vaginal infections; asphyxia; prematurity, and seizures were more common in the autistic group, although the differences were not significant. Thirty-nine percent of the controls, but only 11% of the autistic, group, reported allergies. The increased number of autoimmune disorders shows that in autism, immune dysfunction interacts with various environmental factors to play a role in autism pathogenesis. According to Edelson S. B. and Cantor D. S. (Toxicol. Ind. Health July-August 1998; 14(4):553-63) the advances in medical technology during the last four decades have provided evidence for an underlying neurological basis for autism. The etiology for the variations of neurofunctional anomalies found in the neurological disorder spectrum behaviors appears inconclusive as of this date but growing evidence supports the proposal that chronic exposure to toxic agents, i.e., xenobiotic agents, resulting in a inflammatory reaction directed towards a developing central nervous system may be the best model for defining the physiological and behavioral data found in these populations. Also, an examination of 18 autistic children in blood analyses that were available showed that 16 of these children showed evidence of levels of toxic chemicals exceeding adult maximum tolerance. In the two cases where toxic chemical levels were not found, there was abnormal D-glucaric acid findings suggesting abnormal xenobiotic influences on liver detoxification processes. A proposed mechanism for the interaction of xenobiotic toxins with immune system dysfunction and continuous and/or progressive endogenous toxicity is presented as it relates to the development of behaviors found in the autistic spectrum. Jyonouchi H. et al. (J. Neuroimmunol. Nov. 1, 2001; 120(1-2):170-9) determined innate and adaptive immune responses in children with developmental regression and autism spectrum disorders (ASD, N=71), developmentally normal siblings (N=23), and controls (N=17), and found a clear relationship between proinflammatory and regulatory cytokine production associated with innate and adaptive immune responses in children with autism spectrum disorders and developmental regression. With lipopolysaccharide (LPS), a stimulant for innate immunity, peripheral blood mononuclear cells (PBMCs) from 59/71 (83.1%) ASD patients produced >2 SD above the control mean (CM) values of TNF-α, IL-1β, and/or IL-6 produced by control PBMCs. ASD PBMCs produced higher levels of proinflammatory/counter-regulatory cytokines without stimuli than controls. With stimulants of phytohemagglutinin (PHA), tetanus, IL-12p70, and IL-18, PBMCs from 47.9% to 60% of ASD patients produced >2 SD above the CM values of TNF-α depending on stimulants. These results indicate excessive innate immune responses as a result of NFκB induced cytokine expression in a number of ASD children that is most evident in TNF-α production. Furthermore, according to Messahel S. et al. (Neurosci. Lett. Jan. 23, 1998; 241(1):17-20) the pterins, neopterin and biopterin, occur naturally in body fluids including urine. It is well established that increased neopterin levels are associated with activation of the cellular immune system and that reduced biopterins are essential for neurotransmitter synthesis. It has been also been suggested that some autistic children may be suffering from an autoimmune disorder. To investigate this further the above authors performed high performance liquid chromatography analyses of urinary pterins in a group of pre-school autistic children, their siblings and age-matched control children. Both urinary neopterin and biopterin were raised in the autistic children compared to controls and the siblings showed intermediate values.

As yet another example, the chronic fatigue syndrome (CFS) is a clinically defined condition characterized by severe disabling fatigue and a combination of symptoms that prominently features self-reported impairments in concentration and short-term memory, sleep disturbances, and musculoskeletal pain. Heretofore, the diagnosis of the chronic fatigue syndrome could only be made after other medical and psychiatric causes of chronic fatiguing illness were excluded. No pathognomonic signs or clear diagnostic tests for this condition have yet been validated. Thus far, no definitive treatment exists. Recent longitudinal studies suggest that some persons affected by the chronic fatigue syndrome improve with time but that most remain functionally impaired for several years. CFS is characterized by debilitating fatigue that is not attributable to known clinical conditions, that has lasted for >6 months, that has reduced the activity level of a previously healthy person by >50%, and that has been accompanied by flu-like symptoms (e.g., pharyngitis, adenopathy, low grade fever, myalgia, arthralgia, headache) and neuropsychological manifestations (e.g., difficulty concentrating, exercise intolerance, and sleep disturbances). CFS is frequently of sudden onset. There have been considerable advances in our understanding of the mediators of CFS, with several careful studies of immunologic function, activation, and cytokine dysregulation. An increasing number of independent groups have reported abnormalities of both T and B cell lymphocyte and NK cell function, with one group correlating levels of NK cell function to disease severity. It was suggested that the illness be named chronic immune activation syndrome given the abnormally elevated markers of T cell activation measured on T cells and cytotoxic T cells.

Over the last decade, investigators have demonstrated that individuals with CFS have significantly increased proportions of activated CD8+ T cells, decreased natural killer cell (NK) cytotoxic and lymphoproliferative activities, elevated serum levels of tumor necrosis factor (TNF)-α and β, and detectable TNF-β, interleukin (IL)-1β and IL-6 mRNA in peripheral blood mononuclear cells (PBMC). CFS patients, as a group, also have significantly higher levels, as compared to controls, of soluble TNF receptor type I (sTNF-RI), sIL-6R and β2-microglobulin (β2-m), but not of IL-1 receptor antagonist (IL-1Ra). Correlative and population distribution studies that included lymphoid phenotypic distributions and function as well as soluble immune mediator expression levels revealed the existence of at least two mainly nonoverlapping categories among CFS patients with either: (1) dysregulated TNF-α/β, expression in association with changes in the serum levels of IL-1α, IL-4, sIL-2R, and IL-1Ra, PBMC-associated expression of IL-1β, IL-6, and TNF-β mRNA, and T cell activation; or (2) interrelated and dysregulated expression of sTNF-R1, sIL-6R, and β2-microglobulin and significantly decreased lymphoproliferative and NK cell cytotoxic activities. Furthermore, allostasis—the ability to achieve stability through change—is critical to survival, and many psychological disorders are manifestations of the fact that such stability is not present. Through allostasis, the autonomic nervous system, the hypothalamic-pituitary-adrenal (HPA) axis, and the cardiovascular, metabolic, and immune systems protect the body by responding to internal and external stress. The price of this accommodation to stress can be allostatic load, which is the wear and tear that results from chronic overactivity or underactivity of allostatic systems.

The core of the body's response to a challenge is twofold, turning on an allostatic response that initiates a complex adaptive pathway, and then shutting off this response when the threat is past. The most common allostatic responses involve the sympathetic nervous systems and the HPA axis. For these systems, activation releases catecholamines from nerves and the adrenal medulla and leads to the secretion of corticotropin from the pituitary. The corticotropin, in turn, mediates the release of cortisol from the adrenal cortex. Inactivation returns the systems to base-line levels of cortisol and catecholamine secretion, which normally happens when the danger is past. However, if the inactivation is inefficient, there is overexposure to stress hormones. Over weeks, months, or years, exposure to increased secretion of stress hormones results in a so-called allostatic load and its immunopathophysiologic consequences. It has been shown that allostatic load over a lifetime may cause the allostatic systems to wear out or become exhausted. Frailty in old age is generally seen as a consequence of a worn-out allostatic system. A vulnerable link in the regulation of the HPA axis and cognition is the hippocampal region. Wear and tear on this region of the brain leads to dysregulation of the HPA axis and cognitive impairment. Indeed, some, but not all, of the aging people have impairment of episodic, declarative, and spatial memory and hyperactivity of the HPA axis, all of which can be traced to inflammatory hippocampal damage. Recent data show that similar events occur at a younger age in humans with unexplained mood disorders. In one type of allostatic load inadequate responses by some allostatic systems trigger compensatory increases in others. When one system does not respond adequately to a stressful stimulus, the activity of other systems increases, because the underactive system is not providing the usual counter regulation. For example, if cortisol secretion does not increase in response to stress, secretion of inflammatory cytokines (which are counter regulated by cortisol) increases. The negative consequences of an enhanced inflammatory response are, for example, that the affected subjects are very susceptible to autoimmune and inflammatory disturbances, aggravated often by a genetically determined hyporesponsiveness of the HPA axis.

Also, the months following childbirth are a time when some women are susceptible to serious mood disorders. The illnesses can be resistant to conventional psychiatric treatment methods. Cases of postpartum depression or puerperal psychosis often occur in women with a past history of major depression or bipolar disorder. There has been considerable debate as to whether postpartum psychosis is a discrete diagnostic entity or whether it represents a rapidly evolving psychosis that is a manifestation of an underlying bipolar (or manic-depressive) disorder. To date, existing psychiatric research supports the latter view.

The invention provides a method for the treatment of a subject believed to be suffering from a neurologic disorder, with a specific aim of reducing the frequency, and limit the lasting effects of the psychological manifestations of neuroimmune disease, and in particular the treatment of the inflammatory component of neurological or mood disorders to relieve symptoms that arise from the release of additional pro-inflammatory cytokines, in particular during disease progression, to prevent disability arising from disease progression, and to promote CNS tissue repair. The invention provides a pharmaceutical composition for the treatment of a neurological disorder occurring in a subject, for example in a primate, and a method for the treatment of the disease associated with additional pro-inflammatory cytokine release, for example in a primate comprising subjecting the subject to a signaling molecule according to the invention, preferably to a mixture of such signaling molecules. The invention aims at countering the involvement of cell-mediated immunity in the etiology of neurologic disease, and treating the inflammatory component of neurological disorders by targeting the central role of NFκB-induced cytokine expression. As a consequence of (likely CNS-based) NFκB expression, toxic inflammatory mediators are released, sustaining breakdown of the blood-brain barrier and leading to injury of axons and glia. Nitric oxide might act directly on normal or hypomyelinated axons, transiently blocking conduction and reversibly increasing deficits arising from already compromised pathways. As acute inflammation resolves, pathways are released from nitric oxide-induced physiological conduction block. Symptoms also improve as surviving functional pathways are reorganised at the cellular and systems level. Together, these mechanisms account for remission early in the disease. But tissue vulnerability is easily exposed. When compounded by high axonal firing frequency, nitric oxide causes structural (and hence irreversible) changes to axons. Cytokines and growth-promoting factors released by reactive astrocytes and microglia as part of the acute inflammatory process promote endogenous remyelination. But, over time, astrocyte reactivity seals the lesion and gliosis causes a physical barrier to further remyelination, reducing the capacity to accommodate cumulative deficits, and marking transition to the stage of persistent deficit. Since permanent disability can be caused by incomplete recovery from the inflammation, the invention provides a method for modulating a neurological disorder in a subject believed to be in need thereof comprising providing the subject with a signaling molecule comprising a short, gene regulatory peptide or functional analogue thereof, wherein the signaling molecule is administered in an amount sufficient to modulate the exacerbating event. The signal molecule is preferably a short peptide, preferably at most 30 amino acids long, or a functional analogue or derivative thereof. In a much preferred embodiment, the peptide is an oligopeptide of from about 3 to about 15 amino acids long, preferably 4 to 12, more preferably 4 to 9, most preferably 4 to 6 amino acids long, or a functional analogue or derivative thereof. Of course, such signaling molecule can be longer, for example by extending it (N- and/or C-terminally), with more amino acids or other side groups, which can for example be (enzymatically) cleaved off when the molecule enters the place of final destination, however, by virtue of its small size of smaller than 15, preferably smaller than 9 amino acids, a peptide or functional analogue according to the invention thereof readily crossing the blood brain barrier. Furthermore such a small peptide as provided herein is very stable and has a pharmaceutical half life greater than 4 hours. Herewith, the invention also provides a method of treatment of mood disorders such as cases of postpartum depression or puerperal psychosis and a use of a signal molecule according to the invention for the preparation of a pharmaceutical composition for the treatment of cases of postpartum depression or puerperal psychosis, in particular by at least partly restoring or mimicking the anti-inflammatory activity of the gene-regulatory peptides LQGV and VLPALP and their functional analogues. In particular a method is provided wherein the signaling molecule modulates translocation and/or activity of a gene transcription factor. It is particularly useful when the gene transcription factor comprises an NF-κB/Rel protein or an AP-1 protein. Many of the neurological disorders events as mentioned above involve increased expression of inflammatory cytokines due to activation of NF-κB and AP-1, and in a preferred embodiment the invention provides a method wherein translocation and/or activity of the NF-κB/Rel protein or AP-1 protein is inhibited. In this way, the destruction of brain tissues like the myelin lining of nerves or plaque formation which disrupts the brain which have been found to be significantly based on autoimmune or proinflammatory destruction caused by a dysregulated release of cytokines and chemokines is inhibited by a treatment according to the invention. In one embodiment, the peptide is selected from the group of peptides LQG, AQG, LQGV, AQGV, LQGA, VLPALP, ALPALP, VAPALP, ALPALPQ, VLPAAPQ, VLPALAQ, LAGV, VLAALP, VLAALP, VLPALA, VLPALPQ, VLAALPQ, VLPALPA, GVLPALP, LQGVLPALPQVVC, LPGCPRGVNPVVS, LPGC, MTRV, MTR, VVC. As the, additional expression of inflammatory cytokines is often due to activation of NF-κB and AP-1. Inflammatory cytokines can be expressed by endothelium, perivascular cells and adherent or transmigrating leukocytes, all inducing numerous pro-inflammatory and procoagulant effects. Together these effects predispose to inflammation, thrombosis and hemorrhage. Of clinical and medical interest and value, the present invention provides the opportunity to selectively control NFκB-dependent gene expression in tissues and organs in a living subject, preferably in a primate, allowing upregulating essentially anti-inflammatory responses such as IL-10, and downregulating essentially pro-inflammatory responses such as mediated by TNF-α, nitric oxide (NO), IL-5, IL-6 and IL-1β.

### EXAMPLES

The invention thus provides use of an NFκB regulating peptide or derivative thereof for the production of a pharmaceutical composition for the treatment of neurological disorders, preferably in a primate, and provides a method of treatment of neurological disorders, notably in a primate. It is preferred when the treatment comprises administering to the subject a pharmaceutical composition comprising an NFκB down-regulating peptide or functional analogue thereof. Examples of useful NFκB down-regulating peptides are VLPALPQVVC, LQGVLPALPQ, LQG, LQGV, GVLPALPQ, VLPALP, VVC, MTR and circular LQGVLPALPQVVC. More down-regulating peptides and functional analogues can be found using the methods as provided herein. Most prominent among NFκB down-regulating peptides are VLPALPQVVC, LQGVLPALPQ, LQG, LQGV, and VLPALP. These are also capable of reducing production of NO by a cell. It is herein also provided to use a composition that comprises at least two oligopeptides or functional analogues thereof, each capable of down-regulation NFκB, and thereby reducing production of NO and/or TNF-α by a cell, in particular wherein the at least two oligopeptides are selected from the group LQGV, AQGV and VLPALP, for the treatment of recurring disease seen with neurological disorders. In response to a variety of signals received by the body in the course of the disease, the NFκB/Rel family of transcription factors is activated and form different types of hetero- and homodimers among themselves to regulate the expression of target genes containing κB-specific binding sites. NF-κB transcription factors are hetero- or homodimers of a family of related proteins characterized by the Rel homology domain. They form two subfamilies, those containing activation domains (p65-RELA, RELB, and c-REL) and those lacking activation domains (p50, p52). The prototypical NFκB is a heterodimer of p65 (RELA) and p50 (NF-κB1). Among the activated NFκB dimers, p50-p65 heterodimers are known to be involved in enhancing the transcription of target genes and p50-p50 homodimers in transcriptional repression. However, p65-p65 homodimers are known for both transcriptional activation and repressive activity against target genes. κB DNA binding sites with varied affinities to different NFB dimers have been discovered in the promoters of several eukaryotic genes and the balance between activated NFκB homo- and heterodimers ultimately determines the nature and level of gene expression within the cell. The term “NFκB-regulating peptide” as used herein refers to a peptide or a modification or derivative thereof capable of modulating the activation of members of the NFκB/Rel family of transcription factors. Activation of NFκB can lead to enhanced transcription of target genes. Also, it can lead to transcriptional repression of target genes. NFκB activation can be regulated at multiple levels. For example, the dynamic shuttling of the inactive NFκB dimers between the cytoplasm and nucleus by IκB proteins and its termination by phosphorylation and proteasomal degradation, direct phosphorylation, acetylation of NFκB factors, and dynamic reorganization of NFκB subunits among the activated NFκB dimers have all been identified as key regulatory steps in NFκB activation and, consequently, in NFκB-mediated transcription processes. Thus, an NFκB-regulating peptide is capable of modulating the transcription of pro-inflammatory cytokine genes that are under the control of NFκB/Rel family of transcription factors. Modulating comprises the upregulation or the downregulation of transcription. In a preferred embodiment, a peptide according to the invention, or a functional derivative or analogue thereof is used for the production of a pharmaceutical composition for the treatment of neurological disorders. NFκB regulating peptide can be given alone or concomitantly to other treatments, the peptide (or analogue) concentration preferably being from about 1 to about 1000 mg/l, but the peptide can also been given on its own, for example in a bolus injection. In acute cases, doses of 1 to 5 mg/kg bodyweight, for example every eight hours in a bolus injection or per infusionem until the patient stabilizes, are recommended. For example in cases where large adverse response are expected or diagnosed, it is preferred to monitor cytokine profiles, such as TNF-α, IL-6 or IL-10 levels, in the plasma of the treated patient, and to stop treatment according to the invention when these levels are normal. In a preferred embodiment it is herein provided to provide the patient experiencing a severe and acute bipolar disorder with a bolus injection of NF-κB down-regulating peptide such as AQGV, LQGV or VLPALP at 2 mg/kg and continue the infusion with an NF-κB down regulating peptide such as AQGV, LQGV or VLPALP or a functional analogue thereof at a dose of 1 mg/kg bodyweight for every eight hours. Dosages may be increased or decreased, for example depending on the outcome of monitoring the cytokine profile in the plasma or CSF of the patient. As the, disease progression is dramatically mediated by cytokines and chemokines. For example, the TNF-α family is then highly elevated in CSF. The down regulation or T cell regulation of these cytokines and chemokines can prevent T cell and dendritic cells from reaching the CNS and then further down regulate the proinflammatory response which produces pathology of the brain and spinal cord. This model of migration of cells to the CNS and then the release of proinflammatory cytokines and chemokines is seen in the following and can be treated by a peptide according to the invention through NFκB regulation, the development of T regulator cells, and the intervention of expression early or pregenes such as C-jun or C-erg. For the pathologist, neurological disorders often present as a disorder of the central nervous system, manifesting as acute focal inflammatory demyelination and axonal loss with limited remyelination. Thus, the primary nature of inflammation is undisputed and is central for treatments that modulate the immune system. There are, however, several aspects that limit the therapeutic efficacy of strategies directed against the inflammatory component of the disease. Currently, immune suppression with corticosteroids is unable to specifically stop the inflammatory regimes. Also, the inflammatory forms of neurological disorder, such as described above with autism, which are now epidemic in United States and European studies responds well in part to the use of an NFκB down regulating peptides according to the invention.

In response to a variety of pathophysiological and developmental signals, the NFκB/Rel family of transcription factors is activated and form different types of hetero- and homodimers among themselves to regulate the expression of target genes containing κB-specific binding sites. NF-κB transcription factors are hetero- or homodimers of a family of related proteins characterized by the Rel homology domain. They form two subfamilies, those containing activation domains (p65-RELA, RELB, and c-REL) and those lacking activation domains (p50, p52). The prototypical NFκB is a heterodimer of p65 (RELA) and p50 (NF-κB1). Among the activated NFκB dimers, p50-p65 heterodimers are known to be involved in enhancing the transcription of target genes and p50-p50 homodimers in transcriptional repression. However, p65-p65 homodimers are known for both transcriptional activation and repressive activity against target genes. κB DNA binding sites with varied affinities to different NFB dimers have been discovered in the promoters of several eukaryotic genes and the balance between activated NFκB homo- and heterodimers ultimately determines the nature and level of gene expression within the cell. The term “NFκB-regulating peptide” as used herein refers to a peptide or a modification or derivative thereof capable of modulating the activation of members of the NFκB/Rel family of transcription factors. Activation of NFκB can lead to enhanced transcription of target genes. Also, it can lead to transcriptional repression of target genes. NFκB activation can be regulated at multiple levels. For example, the dynamic shuttling of the inactive NFκB dimers between the cytoplasm and nucleus by IκB proteins and its termination by phosphorylation and proteasomal degradation, direct phosphorylation, acetylation of NFκB factors, and dynamic reorganization of NFκB subunits among the activated NFκB dimers have all been identified as key regulatory steps in NFκB activation and, consequently, in NFκB-mediated transcription processes. Thus, an NFκB-regulating peptide is capable of modulating the transcription of genes that are under the control of NFκB/Rel family of transcription factors. Modulating comprises the upregulation or the downregulation of transcription. In a preferred embodiment, a peptide according to the invention, or a functional derivative or analogue thereof is used for the production of a pharmaceutical composition. Examples of useful NFκB downregulating peptides to be included in such a pharmaceutical composition are VLPALPQVVC, LQGVLPALPQ, LQG, LQGV, GVLPALPQ, VLPALP, VVC, MTR and circular LQGVLPALPQVVC. More gene-regulating peptides and functional analogues can be found in a (bio)assay, such as an NFκB translocation assay as provided herein. Most prominent among NFκB down-regulating peptides are VLPALPQVVC, LQGVLPALPQ, LQG, LQGV, and VLPALP. These are also capable of reducing production of NO by a cell. Furthermore, LQG, VVC and MTRV, and in particular LQGV promote angiogenesis, especially in topical applications.

It is herein also provided to use a composition that comprises at least two oligopeptides or functional analogues thereof, each capable of down-regulation NFκB, and thereby reducing production of NO and/or TNF-α by a cell, in particular wherein the at least two oligopeptides are selected from the group LQGV, AQGV and VLPALP. Useful NFκB up-regulating peptides are VLPALPQ, GVLPALP and MTRV. As indicated, more gene-regulatory peptides may be found with an appropriate (bio)assay. A gene-regulatory peptide as used herein is preferably short. Preferably, such a peptide is 3 to 15 amino acids long, more preferably, wherein the lead peptide is 3 to 9 amino acids long, most preferred wherein the lead peptide is 4 to 6 amino acids long, and capable of modulating the expression of a gene, such as a cytokine, in a cell. In a preferred embodiment, a peptide is a signaling molecule that is capable of traversing the plasma membrane of a cell or, in other words, a peptide that is membrane-permeable.

Functional derivative or analogue herein relates to the signaling molecular effect or activity as for example can be measured by measuring nuclear translocation of a relevant transcription factor, such as NF-κB in an NF-κB assay, or AP-1 in an AP-1 assay, or by another method as provided herein. Fragments can be somewhat (i.e. 1 or 2 amino acids) smaller or larger on one or both sides, while still providing functional activity. Such a bioassay comprises an assay for obtaining information about the capacity or tendency of a peptide, or a modification thereof, to regulate expression of a gene. A scan with for example a 15-mer, or a 12-mer, or a 9-mer, or a 8-mer, or a 7-mer, or a 6-mer, or a 5-mer, or a 4-mer or a 3-mer peptides can yield valuable information on the linear stretch of amino acids that form an interaction site and allows identification of gene-regulatory peptides that have the capacity or tendency to regulate gene expression. Gene-regulatory peptides can be modified to modulate their capacity or tendency to regulate gene expression, which can be easily assayed in an in vitro bioassay such as a reporter assay. For example, some amino acid at some position can be replaced with another amino acid of similar or different properties. Alanine (Ala)-replacement scanning, involving a systematic replacement of each amino acid by an Ala residue, is a suitable approach to modify the amino acid composition of a gene-regulatory peptide when in a search for a signaling molecule capable of modulating gene expression. Of course, such replacement scanning or mapping can be undertaken with amino acids other than Ala as well, for example with D-amino acids. In one embodiment, a peptide derived from a naturally occurring polypeptide is identified as being capable of modulating gene expression of a gene in a cell. Subsequently, various synthetic Ala-mutants of this gene-regulatory peptide are produced. These Ala-mutants are screened for their enhanced or improved capacity to regulate expression of a gene compared to gene-regulatory polypeptide.

Furthermore, a gene-regulatory peptide, or a modification or analogue thereof, can be chemically synthesized using D- and/or L-stereoisomers. For example, a generegulatory peptide that is a retro-inverso of an oligopeptide of natural origin is produced. The concept of polypeptide retro-inversion (assemblage of a natural L-amino acid-containing parent sequence in reverse order using D-amino acids) has been applied successfully to synthetic peptides. Retro-inverso modification of peptide bonds has evolved into a widely used peptidomimetic approach for the design of novel bioactive molecules which has been applied to many families of biologically active peptide. The sequence, amino acid composition and length of a peptide will influence whether correct assembly and purification are feasible. These factors also determine the solubility of the final product. The purity of a crude peptide typically decreases as the length increases. The yield of peptide for sequences less than 15 residues is usually satisfactory, and such peptides can typically be made without difficulty. The overall amino acid composition of a peptide is an important design variable. A peptide's solubility is strongly influenced by composition. Peptides with a high content of hydrophobic residues, such as Leu, Val, Ile, Met, Phe and Trp, will either have limited solubility in aqueous solution or be completely insoluble. Under these conditions, it can be difficult to use the peptide in experiments, and it may be difficult to purify the peptide if necessary. To achieve a good solubility, it is advisable to keep the hydrophobic amino acid content below 50% and to make sure that there is at least one charged residue for every five amino acids. At physiological pH Asp, Glu, Lys, and Arg all have charged side chains. A single conservative replacement, such as replacing Ala with Gly, or adding a set of polar residues to the N- or C-terminus, may also improve solubility. Peptides containing multiple Cys, Met, or Trp residues can also be difficult to obtain in high purity partly because these residues are susceptible to oxidation and/or side reactions. If possible, one should choose sequences to minimize these residues. Alternatively, conservative replacements can be made for some residues. For instance, Norleucine can be used as a replacement for Met, and Ser is sometimes used as a less reactive replacement for Cys. If a number of sequential or overlapping peptides from a protein sequence are to be made, making a change in the starting point of each peptide may create a better balance between hydrophilic and hydrophobic residues. A change in the number of Cys, Met, and Trp residues contained in individual peptides may produce a similar effect. In another embodiment of the invention, a gene-regulatory peptide capable of modulating gene expression is a chemically modified peptide. A peptide modification includes phosphorylation (e.g., on a Tyr, Ser or Thr residue), N-terminal acetylation, C-terminal amidation, C-terminal hydrazide, C-terminal methyl ester, fatty acid attachment, sulfonation (tyrosine), N-terminal dansylation, N-terminal, succinylation, tripalmitoyl-S-Glyceryl Cysteine (PAM3 Cys-OH) as well as famesylation of a Cys residue. Systematic chemical modification of a gene-regulatory peptide can for example be performed in the process of gene-regulatory peptide optimization.

Synthetic peptides can be obtained using various procedures known in the art. These include solid phase peptide synthesis (SPPS) and solution phase organic synthesis (SPOS) technologies. SPPS is a quick and easy approach to synthesize peptides and small proteins. The C-terminal amino acid is typically attached to a cross-linked polystyrene resin via an acid labile bond with a linker molecule. This resin is insoluble in the solvents used for synthesis, making it relatively simple and fast to wash away excess reagents and by-products.

The peptides as mentioned in this document such as LQG, AQG, LQGV, AQGV, LQGA, VLPALP, ALPALP, VAPALP, ALPALPQ, VLPAAPQ, VLPALAQ, LAGV, VLAALP, VLPALA, VLPALPQ, VLAALPQ, VLPALPA, GVLPALP, VVCNYRDVRFESIRLPGCPRGVNPVVSYAVALSCQCAL, RPRCRPINATLAVEKEGCPVCITVNTTICAGYCPT, SKAPPPSLPSPSRLPGPS, LQGVLPALPQVVC, SIRLPGCPRGVNPVVS, LPGCPRGVNPVVS, LPGC, MTRV, MTR, and VVC were prepared by solid-phase synthesis using the fluorenylmethoxycarbonyl (Fmoc)/tert-butyl-based methodology with 2-chlorotrityl chloride resin as the solid support. The side-chain of glutamine was protected with a trityl function. The peptides were synthesized manually. Each coupling consisted of the following steps: (i) removal of the α-amino Fmoc-protection by piperidine in dimethylformamide (DMF), (ii) coupling of the Fmoc amino acid (3 eq) with diisopropylcarbodiimide (DIC)/1-hydroxybenzotriazole (HOBt) in DMF/N-methylformamide (NMP) and (iii) capping of the remaining amino functions with acetic anhydride/diisopropylethylamine (DIEA) in DMF/NMP. Upon completion of the synthesis, the peptide resin was treated with a mixture of trifluoroacetic acid (TFA)/H₂O/triisopropylsilane (TIS) 95:2.5:2.5. After 30 minutes TIS was added until decolorization. The solution was evaporated in vacuo and the peptide precipitated with diethyl ether. The crude peptides were dissolved in water (50-100 mg/ml) and purified by reverse-phase high-performance liquid chromatography (RP-HPLC). HPLC conditions were: column: Vydac TP21810C18 (10×250 mm); elution system: gradient system of 0.1% TFA in water v/v (A) and 0.1% TFA in acetonitrile (ACN) v/v (B); flow rate 6 ml/minute; absorbance was detected from 190-370 nm. There were different gradient systems used. For example for peptides LQG and LQGV: 10 minutes 100% A followed by linear gradient 0-10% B in 50 minutes. For example for peptides VLPALP and VLPALPQ: 5 minutes 5% B followed by linear gradient 1% B/minute. The collected fractions were concentrated to about 5 ml by rotation film evaporation under reduced pressure at 40° C. The remaining TFA was exchanged against acetate by eluting two times over a column with anion exchange resin (Merck II) in acetate form. The elute was concentrated and lyophilized in 28 hours. Peptides later were prepared for use by dissolving them in PBS.

RAW 264.7 macrophages, obtained from American Type Culture Collection (Manassas, Va.), were cultured at 37° C. in 5% CO2 using DMEM containing 10% FBS and antibiotics (100 U/ml of penicillin, and 100 μ/ml streptomycin). Cells (1×10⁶/ml) were incubated with peptide (10 μg/ml) in a volume of 2 ml. After 8 h of cultures cells were washed and prepared for nuclear extracts.

Nuclear extracts and EMSA were prepared according to Schreiber et al. Methods (Schreiber et al. 1989, Nucleic Acids Research 17). Briefly, nuclear extracts from peptide stimulated or nonstimulated macrophages were prepared by cell lysis followed by nuclear lysis. Cells were then suspended in 400 μl of buffer (10 mM HEPES (pH 7.9), 10 mM KCl, 0.1 mM KCL, 0.1 mM EDTA, 0.1 mM EGTA, 1 mM DTT, 0.5 mM PMSF and protease inhibitors), vigorously vortexed for 15 seconds, left standing at 4° C. for 15 minutes, and centrifuged at 15,000 rpm for 2 minutes. The pelleted nuclei were resuspended in buffer (20 mM HEPES (pH 7.9), 10% glycerol, 400 mM NaCl, 1 mM EDTA, 1 mM EGTA, 1 mM DTT, 0.5 mM PMSF and protease inhibitors) for 30minutes on ice, then the lysates were centrifuged at 15,000 rpm for 2 minutes. The supernatants containing the solubilized nuclear proteins were stored at −70° C. until used for the Electrophoretic Mobility Shift Assays (EMSA).

Electrophoretic mobility shift assays were performed by incubating nuclear extracts prepared from control (RAW 264.7) and peptide treated RAW 264.7 cells with a 32P-labeled double-stranded probe (5′ AGCTCAGAGGGGGACTTTCCGAGAG 3′) synthesized to represent the NF-κB binding sequence. Shortly, the probe was end-labeled with T4 polynucleotide kinase according to manufacturer's instructions (Promega, Madison, Wis.). The annealed probe was incubated with nuclear extract as follows: in EMSA, binding reaction mixtures (20 μl) contained 0.25 μg of poly(dI-dC) (Amersham Pharmacia Biotech) and 20,000 rpm of 32P-labeled DNA probe in binding buffer consisting of 5 mM EDTA, 20% Ficoll, 5 mM DTT, 300 mM KCl and 50 mM HEPES. The binding reaction was started by the addition of cell extracts (10 μg) and was continued for 30 minutes at room temperature. The DNA-protein complex was resolved from free oligonucleotide by electrophoresis in a 6% polyacrylamide gel. The gels were dried and exposed to x-ray films.

The transcription factor NF-κB participates in the transcriptional regulation of a variety of genes. Nuclear protein extracts were prepared from LPS and peptide treated RAW264.7 cells or from LPS treated RAW264.7 cells. In order to determine whether the peptide modulates the translocation of NF-κB into the nucleus, on these extracts EMSA was performed. NF-κB WAS present in the nuclear extracts of RAW264.7 cells treated with LPS or LPS in combination with peptide for 4 hours. Here we see that indeed some peptides are able to modulate the translocation of NF-κB since the amount of labeled oligonucleotide for NF-κB is reduced. In this experiment peptides that show the modulation of translocation of NF-κB are: VLPALPQVVC, LQGVLPALPQ, LQG, LQGV, GVLPALPQ, VLPALP, VLPALPQ, GVLPALP, VVC, MTRV, and MTR.

RAW 264.7 mouse macrophages were cultured in DMEM, containing 10% or 2% FBS, penicillin, streptomycin and glutamine, at 37° C., 5% CO₂. Cells were seeded in a 12-wells plate (3×10⁶ cells/ml) in a total volume of 1 ml for 2 hours and then stimulated with LPS (E. coli 026:B6; Difco Laboratories, Detroit, Mich., USA) and/or NMPF (1 microgram/ml). After 30 minutes of incubation plates were centrifuged and cells were collected for nuclear extracts. Nuclear extracts and EMSA were prepared according to Schreiber et al. Cells were collected in a tube and centrifuged for 5 minutes at 2000 rpm (rounds per minute) at 4° C. (Universal 30 RF, Hettich Zentrifuges). The pellet was washed with ice-cold Tris buffered saline (TBS pH 7.4) and resuspended in 400 μl of a hypotonic buffer A (10 mM HEPES pH 7.9, 10 mM KCl, 0.1 mM EDTA, 0.1 mM EGTA, 1 mM DTT, 0.5 mM PMSF and protease inhibitor cocktail (Complete™ Mini, Roche) and left on ice for 15 minutes. Twenty-five micro liter 10% NP-40 was added and the sample was centrifuged (2 minutes, 4000 rpm, 4° C.). The supernatant (cytoplasmic fraction) was collected and stored at −70° C. The pellet, which contains the nuclei, was washed with 50 μl buffer A and resuspended in 50 μl buffer C (20 mM HEPES pH 7.9, 400 mM NaCl, 1 mM EDTA, 1 mM EGTA, 1 mM DTT, 0.5 mM PMSF and protease inhibitor cocktail and 10% glycerol). The samples were left to shake at 4° C. for at least 60 minutes. Finally the samples were centrifuged and the supernatant (nucleic fraction) was stored at −70° C.

Bradford reagent (Sigma) was used to determine the final protein concentration in the extracts. For Electrophoretic mobility shift assays an oligonucleotide representing NF-κB binding sequence (5′-AGC TCA GAG GGG GAC TTT CCG AGA G-3′) was synthesized. Hundred pico mol sense and antisense oligo were annealed and labeled with γ-³²P-dATP using T4 polynucleotide kinase according to manufacture's instructions (Promega, Madison, Wis.). Nuclear extract (5-7.5 μg) was incubated for 30 minutes with 75000 cpm probe in binding reaction mixture (20 microliter) containing 0.5 μg poly dI-dC (Amersham Pharmacia Biotech) and binding buffer BSB (25 mM MgCl₂, 5 mM CaCl₂, 5 mM DTT and 20% Ficoll) at room temperature. The DNA-protein complex was resolved from free oligonucleotide by electrophoresis in a 4-6% polyacrylamide gel (150 V, 2-4 hours). The gel was then dried and exposed to x-ray film. The transcription factor NF-κB participates in the transcriptional regulation of a variety of genes. Nuclear protein extracts were prepared from either LPS (1 mg/ml), peptide (1 mg/ml) or LPS in combination with peptide treated and untreated RAW264.7 cells. In order to determine whether the peptides modulate the translocation of NF-κB into the nucleus, on these extracts EMSA was performed. Peptide signaling molecules are able to modulate the basal as well as LPS induced levels of NF-κB. In this experiment peptides that show the inhibition of LPS induced translocation of NF-κB are: VLPALPQVVC, LQGVLPALPQ, LQG, LQGV, GVLPALPQ, VLPALP, VVC, MTR and circular LQGVLPALPQVVC. Peptide signaling molecules that in this experiment promote LPS induced translocation of NF-κB are: VLPALPQ, GVLPALP and MTRV. Basal levels of NF-κB in the nucleus was decreased by VLPALPQVVC, LQGVLPALPQ, LQG and LQGV while basal levels of NF-κB in the nucleus was increased by GVLPALPQ, VLPALPQ, GVLPALP, VVC, MTRV, MTR and LQGVLPALPQVVC. In other experiments, QVVC also showed the modulation of translocation of NF-κB into nucleus (data not shown).

Further modes of identification of gene-regulatory peptides by NFκB analysis

Cells: Cells will be cultured in appropriate culture medium at 37° C., 5% CO₂. Cells will be seeded in a 12-wells plate (usually 1×10⁶ cells/ml) in a total volume of 1 ml for 2 hours and then stimulated with regulatory peptide in the presence or absence of additional stimuli such as LPS. After 30 minutes of incubation plates will be centrifuged and cells are collected for cytosolic or nuclear extracts.

Nuclear Extracts: Nuclear extracts and EMSA could be prepared according to Schreiber et al. Method (Schreiber et al. 1989, Nucleic Acids Research 17). Cells are collected in a tube and centrifuged for 5 minutes at 2000 rpm (rounds per minute) at 4° C. (Universal 30 RF, Hettich Zentrifuges). The pellet is washed with ice-cold Tris buffered saline (TBS pH 7.4) and resuspended in 400 μl of a hypotonic buffer A (10 mM HEPES pH 7.9, 10 mM KCl, 0.1 mM EDTA, 0.1 mM EGTA, 1 mM DTT, 0.5 mM PMSF and protease inhibitor cocktail (Complete™ Mini, Roche) and left on ice for 15 minutes. Twenty-five micro liter 10% NP-40 is added and the sample is centrifuged (2 minutes, 4000 rpm, 4° C.). The supernatant (cytoplasmic fraction) was collected and stored at −70° C. for analysis. The pellet, which contains the nuclei, is washed with 50 μl buffer A and resuspended in 50 μl buffer C (20 mM HEPES pH 7.9, 400 mM NaCl, 1 mM EDTA, 1 mM EGTA, 1 mM DTT, 0.5 mM PMSF and protease inhibitor cocktail and 10% glycerol). The samples are left to shake at 4° C. for at least 60 minutes. Finally the samples are centrifuged and the supernatant (nucleic fraction) is stored at −70° C. for analysis.

Bradford reagent (Sigma) could be used to determine the final protein concentration in the extracts.

EMSA: For Electrophoretic mobility shift assays an oligonucleotide representing NF-κB binding sequence such as (5′-AGC TCA GAG GGG GAC TTT CCG AGA G-3′) are synthesized. Hundred pico mol sense and antisense oligo are annealed and labeled with γ-³²P-dATP using T4 polynucleotide kinase according to manufacture's instructions (Promega, Madison, Wis.). Cytosolic extract or nuclear extract (5-7.5 μg) from cells treated with regulatory peptide or from untreated cells is incubated for 30 minutes with 75000 cpm probe in binding reaction mixture (20 μl) containing 0.5 μg poly dI-dC (Amersham Pharmacia Biotech) and binding buffer BSB (25 mM MgCl₂, 5 mM CaCl₂, 5 mM DTT and 20% Ficoll) at room temperature. Or cytosolic and nuclear extract from untreated cells or from cells treated with stimuli could also be incubated with probe in binding reaction mixture and binding buffer. The DNA-protein complex is resolved from free oligonucleotide by electrophoresis in a 4-6% polyacrylamide gel (150 V, 2-4 hours). The gel is then dried and exposed to x-ray film. Peptides can be biotinylated and incubated with cells. Cells are then washed with phosphate-buffered saline, harvested in the absence or presence of certain stimulus (LPS, PHA, TPA, anti-CD3, VEGF, TSST-1, VIP or know drugs etc.). After culturing cells are lysed and cells lysates (whole lysate, cytosolic fraction or nuclear fraction) containing 200 micro gram of protein are incubated with 50 miroliter Neutr-Avidin-plus beads for 1 hour at 4° C. with constant shaking. Beads are washed five times with lysis buffer by centrifugation at 6000 rpm for 1 minute. Proteins are eluted by incubating the beads in 0.05 N NaOH for 1 minute at room temperature to hydrolyze the protein-peptide linkage and analyzed by SDS-polyacrylamide gel electrophoresis followed by immunoprecipitated with agarose-conjugated anti-NF-κB subunits antibody or immunoprecipitated with antibody against to be studied target. After hydrolyzing the protein-peptide linkage, the sample could be analyzed on HPLS and mass-spectrometry. Purified NF-κB subunits or cell lysate interaction with biotinylated regulatory peptide can be analyzed on biosensor technology. Peptides can be labeled with FITC and incubated with cells in the absence or presence of different stimulus. After culturing, cells can be analyzed with fluorescent microscopy, confocal microscopy, flow cytometry (cell membrane staining and/or intracellular staining) or cells lysates are made and analyzed on HPLC and mass-spectrometry. NF-κB transfected (reporter gene assay) cells and gene array technology can be used to determine the regulatory effects of peptides.

HPLC and mass-spectrometry analysis: Purified NF-κB subunit or cytosolic/nuclear extract is incubated in the absence or presence of (regulatory) peptide is diluted (2:1) with 8 N guanidinium chloride and 0.1% trifluoroacetic acid, injected into a reverse-phase HPLC column (Vydac C18) equilibrated with solvent A (0.1% trifluoroacetic acid), and eluted with a gradient of 0 to 100% eluant B (90% acetonitrile in solvent A). Factions containing NF-κB subunit are pooled and concentrated. Fractions are then dissolved in appropriate volume and could be analyzed on mass-spectrometry.

Further references: PCT International Publications WO99/596 17, WO97/49721, WO01/10907, and WO01/11048, the contents of the entirety of all of which are incorporated by this reference.

What is claimed is:

1. A method for modulating a neurological disorder in a subject, said method comprising:
providing said subject with a gene-regulatory peptide or functional analogue thereof.

2. The method according to claim 1 wherein said gene-regulatory peptide or functional analogue thereof down-regulates translocation, activity, or translocation and activity of a gene transcription factor.

3. The method according to claim 2 wherein said gene transcription factor comprises an NF-kappaB/Rel protein.

4. The method according to claim 3 wherein translocation and/or activity of said NF-kappaB/Rel protein is inhibited.

5. The method according to claim 1 wherein said gene-regulatory peptide or functional analogue thereof has NFkappaB down-regulating activity in LPS stimulated RAW264.7 cells.

6. The method according to claim 2 wherein said gene-regulatory peptide or functional analogue thereof has NFkappaB down-regulating activity in LPS stimulated RAW264.7 cells.

7. The method according to claim 3 wherein said gene-regulatory peptide or functional analogue thereof has NFkappaB down-regulating activity in LPS stimulated RAW264.7 cells.

8. The method according to claim 4 wherein said gene-regulatory peptide or functional analogue thereof has NFkappaB down-regulating activity in LPS stimulated RAW264.7 cells.

9. The method according to claim 1 wherein the subject is presenting clinical signs of autism.

10. The method according to claim 2 wherein the subject is presenting clinical signs of autism.

11. The method according to claim 3 wherein the subject is presenting clinical signs of autism.

12. The method according to claim 4 wherein the subject is presenting clinical signs of autism.

13. The method according to claim 5 wherein the subject is presenting clinical signs of autism.

14. The method according to claim 6 wherein the subject is presenting clinical signs of autism.

15. The method according to claim 7 wherein the subject is presenting clinical signs of autism.

16. The method according to claim 8 wherein the subject is presenting clinical signs of autism.

17. The method according to claim 9 wherein said gene-regulatory peptide or functional analogue thereof has NFkappaB down-regulating activity in LPS un-stimulated RAW264.7 cells.

18. The method according to claim 1 wherein said gene-regulatory peptide or functional analogue thereof has NFkappaB down-regulating activity in LPS un-stimulated RAW264.7 cells.

19. The method according to claim 2 wherein said gene-regulatory peptide or functional analogue thereof has NFkappaB down-regulating activity in LPS un-stimulated RAW264.7 cells.

20. The method according to claim 3 wherein said gene-regulatory peptide or functional analogue thereof has NFkappaB down-regulating activity in LPS un-stimulated RAW264.7 cells.
